(19) United States
(12) Patent Application Publication
(10) Pub. No.: US 2016/0184291 A1
(43) Pub. Date: Jun. 30, 2016

(54) TREATMENT FOR COLLAPSING TRACHEA

(71) Applicant: Howard Brooks-Korn, Pacifica, CA (US)

(72) Inventor: Howard Brooks-Korn, Pacifica, CA (US)

(21) Appl. No.: 14/583,761

(22) Filed: Dec. 28, 2014

(51) Int. Cl. A61K 31/485

(52) U.S. Cl. CPC A61K 31/485

(57) ABSTRACT

The present invention provides pharmaceutical methods and formulations for treating collapsing trachea and related disorders.

### CROSS-REFERENCE TO RELATED APPLICATION

This application claims priority from co-pending provisional application U.S. Ser. No. 61/921,416 filed Dec. 28, 2013, entitled “TREATMENT FOR COLLAPSING TRACHEA”.

### FIELD OF THE INVENTION

This invention relates to the treatment of animals suffering from collapsing trachea and related disorders by the administration of an opioid active agent.

### BACKGROUND OF THE INVENTION

“Collapsing trachea,” also referred to as “tracheal choldromalacia,” is a progressive, degenerative condition involving the cartilaginous tracheal rings, the trachealis muscles and the tracheal membrane, resulting in collapse of the tracheal lumen. The symptoms typically manifest as a dry cough often referred to as a “honking” cough as air expelled through the collapsing tracheal lumen during a cough forces the lumen with its attached cartilage to vibrate. As coughing continues, irritation, inflammation and additional stresses cause additional tracheal collapse. The more the cough continues the more the trachea collapses, and the more the trachea collapses the more the animal coughs; a truly vicious cycle.

Typical initial treatments are palliative, aimed at stopping the persistent cough. Over-the-counter cough medicines with dextromethorphan may initially work. As the cough worsens, bronchodilators can be given to open the bronchial airways and decrease airway pressure. Over time, when such treatments prove ineffective, stronger anti-tussive compounds are used (e.g., codeine, hydrocodone and eventually butorphenol). As the collapsing trachea becomes progressively weaker, the cough becomes intractable, every medical treatment failing to stop it, and the patient is euthanized before dying of suffocation. If an animal owner chooses to pursue more aggressive treatment before the tracheal collapse reaches end-stage, a stent can be surgically placed in the trachea to help hold open the area of collapse. The placing of surgical stents can be successful, especially in the cervical trachea. Stents are rarely used in the thoracic trachea due to a high rate of complications and failure. If the stenting procedure fails, euthanasia almost invariably follows.

U.S. Pat. No. 8,410,129 discloses the use of opiates for treatment of centrally and/or peripherally mediated neuropathic and polyneuropathic, disorders and syndromes including but not limited to lingual, pharyngeal, laryngeal, esophageal, urinary bladder sphincter, lumbar and lumbo-sacral spine, pelvis and pelvic-limb paresis/paralysis. This patent reports the successful reversal of paresis/paralysis in such disorders and syndromes, particularly by administering immediate or sustained release pharmaceutical formulations of hydrocodone, oxycodone and morphine sulfate, and teaches the similar use of other opioids. Particularly preferred among the opioids employed in the '129 patent is sustained release oxycodone (OxyContin®) provided in the original formulation, e.g., as taught in U.S. Pat. No. 5,508,042, which expired on Apr. 16, 2013.

Co-pending U.S. applications Ser. Nos. 11/395,200 and Ser. No. 11/395,242 further elucidate the '129 patent's teachings, particularly in view of the premise that abnormal concentrations of one or more endogenous opioids, or the blockade, under-expression or over-expression of one or more opioid receptors, can represent an underlying etiology of an ailment requiring treatment. Also disclosed are veterinary formulations responsive to concerns about diversion, including a “detractant” ingredient, for example, an odor, flavor, texture or other ingredient that while acceptable to a non-human mammal is unacceptable to a human being.

It remains desired to provide an effective, long-term non-surgical (pharmaceutical) treatment for collapsing trachea, particularly for collapsing trachea that has progressed to being refractory or end-stage.

### SUMMARY OF THE INVENTION

One aspect of the present invention provides methods of treatment, particularly a method for treating collapsing trachea by administering to a subject in need thereof (e.g., a dog) an effective amount of oxycodone, particularly oxycodone sustained release. The treatment is also effective for end-stage or refractory collapsing trachea. Another aspect of the invention provides such methods of treatment including the co-administration of low dose naltrexone.

### DETAILED DESCRIPTION OF THE INVENTION

Before describing the present invention in detail, it is to be understood that the terminology used herein is for the purpose of describing particular embodiments only, and is not intended to be limiting.

### Definitions

As used in the present specification, the following words and phrases are generally intended to have the meanings as set forth below, except to the extent that the context in which they are used indicates otherwise. The singular form is intended to include the plural unless the context clearly dictates otherwise. For example, the term “an opioid compound” encompasses one or more opioids, as well as mixtures thereof.

“Extended release” or “sustained release” is defined for purposes of the present invention as the rate at which a drug must be released after administration in order to maintain a therapeutically effective blood (plasma) level for a minimum of about 6 hours, but preferably lasting 12 to 36 hours or longer. This term is also employed to identify a formulation having such a release profile.

“Immediate release” is defined for purposes of the present invention as the rate at which a drug must be released after administration in order to maintain a therapeutically effective blood (plasma) level for period of about 6 hours or less. This term is also employed to identify a formulation having such a release profile.

By “therapeutically effective amount” is meant a nontoxic amount of a drug, active agent or formulation in a quantity sufficient to provide a desired therapeutic effect, e.g., treatment of an ailment.

Collapsing trachea is a progressive, degenerative disease predominantly found in older, small and toy-breed dogs (e.g., Chihuahua, Shiatsu, Lhasa Apso, Maltese, Pug, Pomeranian, Miniature Poodle, Toy Poodle, and Yorkshire Terrier). It has also been reported in larger breed dogs and in horses as well.

The trachea connects the distal end of the larynx to the main stem bronchus, where the bronchial tubes branch off from the end of the trachea and extend into the lungs allowing inspired air to pass from the nose or mouth, through the oral or nasal pharynx through the arytenoids and larynx into the trachea and bronchial tubes and into the lungs, and then back out again. The trachea is comprised of aligned, hyaline cartilaginous ‘C’-shaped rings. Each tracheal ring is covered by the trachealis muscle, including the open end of the aligned C's, where it is referred to as the tracheal membrane. This sheet of muscle not only allows for a small amount of play in the size of the cartilaginous rings but also covers the inner lining of the trachea. In a healthy trachea the diameter of tracheal lumen stays the same throughout all the phases of respiration.

In collapsing trachea, apparent weakening of the hyaline cartilage rings and trachealis muscle causes a loss of rigidity. The trachea fails to maintain its open shape, losing diameter during respiration. Increased tension on, or neurogenic atrophy of, the trachealis dorsalis muscle causes stretching of the dorsal tracheal membrane resulting in protrusion of the tracheal membrane into the tracheal lumen. Breathing exacerbates the dorsal/ventral cartilage collapse narrowing the lumen to the point where the tracheal membrane can touch the tracheal lining, causing a tickling sensation (simulating the perception of a foreign body or mucous) triggering the cough reflex. Symptoms eventually worsen until the typically identified dry, honking cough (or “goose honk”) is heard as air expelled during expiration through the collapsing tracheal lumen forces the lumen with its attached cartilage to vibrate. Other symptoms include exercise intolerance (especially if the dog is walked using a normal collar and leash), mucosal inflammation, swelling, respiratory distress, and right sided cardiac enlargement. Eventually the unstoppable coughing cuts off the supply of oxygen to the lungs and results in suffocation.

Typical initial treatments are palliative (such as use of over-the-counter cough medicines with dextromethorphan and prescription bronchodilators). Over time such treatments become ineffective, so stronger anti-tussive compounds are used (e.g., codeine cough suppressants and butorphenol). Eventually as the disease progresses the trachea becomes so pliable that even these medications become ineffective and the collapsing trachea reaches its refractory or end-stage condition. At this point the pet must be euthanized before it suffocates.

Recently, surgical stent placement (which helps hold the tracheal rings open) has become more successful. The placing of surgical stents is unfortunately restricted to the extra thoracic, cervical trachea and still has a significant number of complications. One surgical procedure still remains an option and that is to make a permanent tracheotomy opening in the neck, which will only work if it can be placed below the level of the collapsed trachea. Unfortunately many of the dogs affected also have a problem with collapse of the Corina, the terminal end of the trachea where it joins the main stem bronchi. A tracheotomy will not work in this case and again euthanasia is the only recourse.

While not intending to be limited by any particular mechanism of action, it is believed that at least part of the disease is due to failure of the musculature supporting the trachea, potentially through loss of neuromuscular signaling. My previous work in the treatment of Upper Respiratory Obstructive Syndrome (including lingual, pharyngeal, laryngeal, and/or esophageal paresis/paralysis) has shown that the administration of opiates, particularly oxycodone, can restore function to apparently paralyzed muscles. It has surprisingly been found that oxycodone can be administered to treat collapsing trachea, even in its refractory or end-stage where strong antitussives such as butorphenol have stopped working.

Thus, the present invention provides an effective, long-term treatment for collapsing trachea, which can be effective throughout the disease, providing treatment when the disease progresses to the refractory or end stage where all other treatments have stopped working. Moreover, results observed with oxycodone sustained release exceed those obtained with other treatments; not only does the cough resolve but improvement is also seen in other symptoms including exercise intolerance, panting and respiratory distress. Thus the present invention offers an effective long-term treatment for collapsing trachea at all stages of the disease.

The active agent used in the present invention is a kappa opioid agonist, preferably oxycodone (sustained or immediate release, preferably sustained release).

The amount of active agent administered will depend on the age and general condition of the subject being treated, the severity of the subject's condition, the dosing regimen and the judgment of the prescribing veterinarian. While daily dosing regimens can involve the administration of as many as eight doses, it is generally preferred that the number of doses be kept to a minimum for example to facilitate patient compliance. Moreover, the longer acting sustained-release dosage forms maintain a more consistent plasma concentration of the active agent, avoiding peaks and troughs.

For an animal subject weighing in the range of about 2.5 kg to about 10 kg, such as a dog, a starting dose of oxycodone sustained release can be about 2.5 mg to about 10 mg given every 12 hours. The dose of medication is adjusted according to the weight and need of a subject, to ameliorate the presenting symptoms. One of ordinary skill in the art will have the experience and means to determine the adjustment needed. Typically, if the symptoms have not started to resolve after about 2 to 24 hours of treatment (or if a subject starts to show the symptoms of drug tolerance) the dose is elevated by a factor ranging from 1.25 to 2.0 times the original dosage (preferably 1.5 times the original dose, e.g., 10 mg twice daily is increased to 15 mg twice daily) escalating on about a 48 hour basis until the symptoms are effectively treated at a well-tolerated dosage. Typically, the maximum dose of oxycodone sustained release administered to large dogs has been 40 mg twice daily. Giant breed dogs in the range of sixty kilograms and up can start at 40 mg, but may need to use 60 to 80 mg of sustained release oxycodone twice daily.

Concomitant treatment with low doses of naltrexone is also advised to prevent the onset of tolerance to the oxycodone.

The lowest therapeutically effective dosage is the least amount of the pharmaceutical formulation sufficient to effect treatment. The highest tolerated dosage is the maximum amount of the pharmaceutical formulation to effect treatment without the occurrence of adverse side effect(s) outweighing the benefit received. If the subject's highest tolerated dosage is reached, but the symptoms are not abated, treatment should be discontinued, optionally re-commencing treatment by substituting a different active agent or formulation.

Use of a diversion-resistant canine veterinary formulation is preferred, one example being the hard-coated tablets currently used for rendering Oxycontin uncrushable to prevent diversion by injection or inhalation. Another such formulation includes an active agent that has the potential for diversion and abuse by humans, and an anti-diversive ingredient that is unacceptable to humans yet acceptable to dogs, for example if they are less sensitive to it than humans. This can be a smell or a flavor or an active ingredient, for example present in a human effective amount and less than an animal effective amount and selected from the group: ipecac, emetine, cephaeline, CCK8 and copper sulphate.

One such diversion-resistant canine veterinary formulation includes: an active agent that has the potential for diversion and abuse by humans, and ipecac in an amount sufficient to cause nausea and/or emesis in a human but insufficient to cause nausea and/or emesis in a dog. The diversion-resistant canine veterinary formulation can include from about 0.07 mg to about 7.0 mg of ipecac, preferably formulated for immediate release. Where the active agent that has the potential for diversion and abuse by humans is oxycodone, the formulation can include from about 2.5 mg to about 40 mg of oxycodone, preferably formulated for sustained release. The ipecac in such formulations can be formulated for release below a pH in the range of about 1.6 to 2.0.

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

### Example 1

### Treatment of Collapsing Trachea

An 8 year old, spayed female Pomeranian weighing 9.3 pounds presented with a history of collapsing trachea that had been responsive to treatment with Torbutrol (butorphenol), which had been controlling the cough but leaving the patient highly intolerant to exercise (to the point of having to be carried) and continuously panting. Upon presentation, the patient had severe dyspnea and coughing. X-ray of the oral pharynx and chest confirmed a diagnosis of severe intrathoracic collapsing trachea, in the refractory stage having stopped responding to Torbutrol.

Oxycodone sustained release was prescribed, 10 mg twice daily. While the patient's cough did resolve, after 5 days lethargy and a loss of appetite were observed to be side effects of opioid overdose, so treatment was discontinued. When appetite returned (along with resumed coughing) the patient's dosage was adjusted to 2.5 mg twice daily (cutting the 10 mg tablets in quarters). This lower dose treatment was effective in resolving the patient's cough and was more suitable for the patient's size. Additionally, the patient has resumed running and playing without coughing or difficulty breathing. On two occasions when the beginning of a cough was observed, a single additional 2.5 mg tablet was administered, followed by return to the original twice-daily dosing regimen. The treatment has been effective for over one year since initiation. Efficacy in this treated dog has been confirmed by radiographic examination.

### Examples 2 and 3

### Treatment of Collapsing Trachea

The treatment described in Example 1 has been successfully repeated in two additional dogs diagnosed as having collapsing trachea.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications (including websites) cited above are hereby incorporated by reference.

I claim:

1. A method for treating collapsing trachea by administering to a subject in need thereof an effective amount of a kappa opioid agonist.

2. The method for treating collapsing trachea of claim 1 wherein the kappa opioid agonist is oxycodone.

3. A method for treating collapsing trachea by administering to a subject in need thereof an effective amount of oxycodone.

4. A veterinarily acceptable composition for treating collapsing trachea comprising an effective amount of a kappa opioid agonist.

5. The veterinarily acceptable composition for treating collapsing trachea of claim 4 wherein the kappa opioid agonist is oxycodone.
